(19) United States
(12) Patent Application Publication
(10) Pub. No.: US 2026/0283476 A1
(43) Pub. Date: Sep. 24, 2026

(54) Systems and Methods for Detecting and Documenting Cryogenic Therapy of a Patient

(71) Applicant: Katherine Bao-Shian Lee MD, APC, Laguna Hills, CA (US)

(72) Inventor: Katherine Lee, Laguna Hills, CA (US)

(73) Assignee: Katherine Bao-Shian Lee MD, APC, Laguna Hills, CA (US)

(21) Appl. No.: 19/683,608

(22) Filed: May 20, 2026

Related U.S. Application Data
(63) Continuation-in-part of application No. 19/288,633, filed on Aug. 1, 2025.
(60) Provisional application No. 63/707,079, filed on Oct. 14, 2024.
(60) Provisional application No. 63/805,179, filed on May 13, 2025.

(51) Int. Cl. A61B 5/01; A61B 5/00; A61B 18/02; G16H 10/60; G16H 30/40

(52) U.S. Cl. CPC A61B 5/015; A61B 5/0013; A61B 5/0077; G16H 10/60; G16H 30/40; A61B 18/02

(57) ABSTRACT

A medical imaging and documentation system captures visible spectrum image data and thermal spectrum image data associated with a body surface of a patient. One or more processors determine a site representation corresponding to a site of interest, map the site representation to an anatomic coordinate space, and store an anatomic localization record in association with a patient record. This can be used to accurately document treatment locations in a patient's file and identify the treatment locations or locations of a site of interest during a subsequent patient encounter.

This application is a continuation-in-part application of U.S. non-provisional patent application having Ser. No. 19/288,633 filed on Aug. 1, 2025, which itself claims priority to U.S. provisional patent application having Ser. No. 63/707,079 filed on Oct. 14, 2024, and U.S. provisional patent application having Ser. No. 63/805,179 filed on May 13, 2025. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is medical imaging systems and methods, and in particular, systems and methods for documenting dermatologic procedures, mapping treatment sites to anatomic coordinates, and guiding future medical procedures using stored anatomic localization data.

### BACKGROUND

The following description includes information that may be useful in understanding the present invention. It is not an admission that any of the information provided herein is prior art or relevant to the presently claimed invention, or that any publication specifically or implicitly referenced is prior art.

Cryogenic therapy (cryotherapy) represents a cornerstone treatment modality in dermatology, utilizing liquid nitrogen to induce rapid freezing and controlled destruction of abnormal skin tissue. This technique is routinely employed for managing various cutaneous conditions including, for example, warts, skin tags, actinic keratoses, seborrheic keratoses, and small superficial basal cell carcinomas. A provider may treat several distinct sites during a single patient encounter.

Medical providers need to document each patient visit into that patient's electronic medical record (EMR) to facilitate receiving reimbursement and recording the visit for compliance. This documentation, often referred to as a medical note or notation, may include both written notations from the provider and/or images or information from diagnostics or tests. In the specific case of cryotherapy, typical clinical practice involves treatment of between 10-30 or more distinct anatomical sites per patient encounter.

Conventional documentation of the treated sites may rely on manual charting and verbal dictation. Many providers use a scribe who manually adds the treated locations, clinical diagnoses, and/or procedural details in the medical record, or the provider themselves needs to document the treatment on their own after the completed procedure. This standard of care is not only inefficient, but ineffective, as it relies on a secondary person or the provider's memory to chart the treated areas. These inefficient methods and tools increase costs and obligations on the provider, and generally reduce accuracy of the recorded/notated medical documentation of the EMR.

A provider may need to identify a previously biopsied site during a later patient encounter. For example, a malignant or precancerous lesion may be biopsied during a first visit and surgically removed during a later visit after the biopsy site has partially or fully healed. A provider may also need to confirm the exact location of a new skin lesion, such as a mole, relative to prior images, or assign an anatomic location to a skin lesion or skin condition for future monitoring or treatment. Existing photographic records may not reliably relocate such sites.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Thus, there is still a need for thermal and visual medical imaging systems and methods for treatment site documentation and anatomic localization, which allow a medical provider to accurately notate a patient's electronic medical record, and in particular, to update a patient's electronic medical record with treatment locations or locations of anomalies.

### SUMMARY OF THE INVENTION

The inventive subject matter provides apparatus, systems, and methods for more efficient and effective tools for a medical provider to accurately, and preferably automatically, notate a patient's electronic medical record with documentation of skin anomalies and/or cryotherapy treatment including, for example, treatment locations, clinical diagnoses, and/or procedural details. The inventive subject matter addresses the inefficiencies discussed above by utilizing an integrated hardware-software solution that leverages thermal imaging technology and an advanced machine learning model to automate the documentation and charting processes.

Embodiments provide a system comprising an image capture device, a processor, a memory, and a display. The image capture device preferably captures visible spectrum image data and thermal spectrum image data associated with a body surface of a patient. The processor detects a treatment site, lesion site, biopsy site, or other site of interest using one or more of the visible spectrum image data and the thermal spectrum image data. The processor maps the site of interest to an anatomic coordinate space and stores an anatomic localization record including stored anatomic coordinates for the site of interest for the patient. The display presents documentation or guidance information based on the stored anatomic coordinates.

In some embodiments, the anatomic coordinate space includes a standardized body coordinate space, a patient-specific body coordinate space, a two-dimensional body map, a three-dimensional body mesh, a surface parameterization, a body region identifier, a coordinate pair, or combination(s) thereof. The stored anatomic coordinates can be used to review locations where treatment occurred, conduct treatment on specific areas of the body in a future encounter, guide a provider to a corresponding anatomic location, confirm an original biopsy site location before surgical removal of a malignant or precancerous lesion, confirm the exact location of a new skin lesion, or assign an anatomic location to a skin lesion or condition.

The tools of the inventive subject matter described herein uniquely allow for the capture and creation of composite imaging of thermal and visible spectra, preferably using a portable interface. The inventive subject matter further utilizes these tools in methods, applications, interfaces, and systems. Moreover, these tools and related methods, application, interfaces, and systems advantageously eliminate the requirement for a secondary recording person or otherwise manually notation of cryogenic therapy on a patient's electronic medical record.

Contemplated systems and methods for documenting cryogenic therapy of a patient utilize or comprise a thermal spectrum camera (such as a FLIR™ thermal imaging camera or other suitable image capture device) configured to generate a thermal spectrum first image (e.g., a first image) and a visible spectrum camera configured to generate a visible spectrum image (e.g., a second image). Of course, videography could also be used, where a video is captured and analyzed instead of one or more images.

A server is preferably configured to receive the first image and the second image, videos, or information related thereto, which can then be aggregated by the server to identify a set of treatment locations on the patient. Alternatively, a portable computing device (image capture device) comprising the thermal spectrum camera and the visible spectrum camera may utilize specialized software components to capture thermal and visual imagery and aggregate those images before transmitting them to the server.

The identified treatment locations can be transmitted to a medical record system to document the treatment locations in the patient's medical record. The treatment locations can be used to update a patient's medical record by notating the treatment locations on the medical record and/or could be saved as a pdf or other document file in the patient's file for future reference.

Contemplated methods of documenting cryogenic therapy of a patient comprise receiving a thermal spectrum image and a visible spectrum image of a patient. The thermal spectrum image can be aggregated with the visible spectrum image to create a composite spectrum image. A set of treatment locations on the patient can then be identified by analyzing thermal spectrum image, the visible spectrum image, and/or the composite spectrum image. The set of treatment locations are transmitted to a medical record system to automatically document the treatment locations in a medical record of the patient.

Applications are also contemplated for documenting cryogenic therapy of a patient comprising a non-transitory machine-readable medium having instructions stored thereon for execution by a processor to perform a method. The method comprises storing a thermal spectrum image captured by a thermal spectrum camera on a server and storing a visible spectrum image captured by a visible spectrum camera on the server.

The method further comprises aggregating the thermal spectrum image and the visible spectrum image using a processor of the server to create a composite spectrum image, by aligning identical points of the thermal spectrum image with those of the visible spectrum image such that the overlapping images create the composite spectrum image. Areas on the composite spectrum image can be detected that are cooler than surrounding areas using the processor. This can be done using a machine learning model trained to automatically detect treatment sites and utilize anatomical mapping to determine locations of the detected treatment sites.

For example, each of the treatment sites can be assigned a location marker based on its location relative to the patient using the processor. The server can interface with a medical record database of a medical record system. The location markers can be transferred or transmitted to the medical record database of the EMR system, such that the medical record of the patient is automatically notated with the treatment sites.

Another aspect of the inventive subject matter comprises a composite spectrum image capture tool for synchronous capture of thermal and visible spectra images of a patient. The composite spectrum image can be captured using a thermal spectrum camera and a visible spectrum camera, which can be triggered mechanically (e.g., clicking a button or other actuation) or by software to capture the respective images. While it is preferred that the images are captured synchronously, it is also contemplated that the images may be automatically captured sequentially once the button is pressed, for example. As discussed above, the images can be aggregated to create a composite spectrum image that can be stored on a machine-readable medium.

This dual-modality approach of the inventive subject matter enables precise temperature measurement at treatment sites using the thermal imaging, preferably with a sensitivity of approximately 0.05° C., providing objective quantification of freezing and treatment intensity that was previously impossible with traditional clinical documentation methods.

The inventive subject matter provides a significant advancement in dermatological practice with several important clinical implications. For example, enhanced documentation accuracy can be achieved by automatically detecting and mapping treatment sites with precise anatomical localization. This allows the system to reduce reliance on manual documentation processes that are susceptible to human error and inconsistency. In addition, automation of documentation processes reduces administrative burden, potentially allowing clinicians to focus more attention on patient care rather than record-keeping. Comprehensive treatment data enables systematic analysis of clinical patterns and outcomes, potentially informing best practices for cryotherapy application.

In some embodiments, contemplated systems comprise a mobile capture application that operates at a point of care and a review application that operates after image capture. The mobile capture application may provide patient record lookup, encounter creation, real-time visible and thermal image visualization, local storage, authentication, encryption, offline operation, and subsequent synchronization with a server. The review application may provide synchronized review of visible spectrum images and thermal spectrum images, treatment site confirmation, lesion annotation, body map review, encounter finalization, electronic medical record export, role-based access control, and audit logging.

In some embodiments, contemplated methods comprise capturing a visible spectrum image and a thermal spectrum image during or after a dermatologic procedure, identifying a site of interest in at least one of the images, registering the visible spectrum image and the thermal spectrum image, mapping the site of interest to an anatomic coordinate space, storing an anatomic localization record including coordinates of the site of interest, and using the anatomic localization record during a subsequent encounter to display a location indicator corresponding to the site of interest.

A medical imaging and documentation system captures visible spectrum image data and thermal spectrum image data associated with a patient. A processor detects a treatment site, biopsy site, lesion site, or other site of interest, registers image data across imaging modalities or across encounters, and maps the site of interest to an anatomic coordinate space. Stored anatomic coordinates are used to document where treatment occurred, review treatment history, re-identify a biopsy or lesion site in a later image, assign an anatomic location to a lesion or condition, and guide a future surgical, cryotherapy, topical, biopsy, or monitoring procedure and/or used to precisely compare facial features such as wrinkles or sun spots after treatment. The system may provide real-time temperature information, threshold-based capture notifications, audio-triggered capture, duplicate-site resolution, longitudinal lesion tracking, review workflows, and electronic medical record export.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 illustrates a schematic view of one embodiment of a system for documenting cryogenic therapy of a patient.

FIGS. 2A-2D illustrates various views of one embodiment of an image capture device mounted to a cryotherapy treatment applicator.

FIG. 3 illustrates a flowchart of one embodiment of a method for documenting cryogenic therapy of a patient.

FIG. 4 illustrates a schematic of a process to map two-dimensional treatment site coordinates to standardized UV space.

FIG. 5 illustrates a flowchart of a process for automatically identifying and mapping treatment site locations on a patient.

FIG. 6A illustrates an exemplary visible spectrum image.

FIG. 6B illustrates an exemplary thermal spectrum image.

FIG. 6C illustrates an exemplary composite spectrum image.

FIG. 7 illustrates an exemplary chart of a patient's medical record notating treatment locations on the patient.

FIG. 8 is a schematic view of certain embodiments of systems of the present invention.

FIG. 9 is a schematic view of certain embodiments of methods of the present invention.

FIG. 10 illustrates a schematic view of another embodiment of a medical imaging and documentation system.

FIGS. 11A-11C illustrates an example workflow for mapping a detected site to an anatomic coordinate space, detecting duplicate treatment sites, and re-identifying site locations.

### DETAILED DESCRIPTION

Throughout the following discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

Embodiments of the inventions described herein may include or utilize a special purpose or general-purpose computer that includes one or more servers and/or other computer hardware. The one or more servers can each include, for example, one or more processors and system memory. The computer can also include physical and other computer-readable media for carrying or storing computer-executable instructions and/or data structures. Such instructions can facilitate the systems and methods described and may be stored in a non-transitory computer-readable medium and executable by the one or more servers or other computing devices. As an example, a processor may receive instructions from a non-transitory computer-readable medium and execute those instructions to perform one or more processes.

Computer-readable media can be any available media that can be accessed by a general purpose or special purpose computer system. Examples of computer-readable media include RAM, ROM, EEPROM, solid state drives, Flash memory, and other optical disk storage, magnetic disk storage or other magnetic storage devices, or any other medium which can be used to store desired application code in the form of computer-executable instructions or data structures, and which can be accessed by a general purpose or special purpose computer.

Computer-executable instructions include, for example, instructions and data which, when executed at a processor, cause a general-purpose computer, special purpose computer, or special purpose processing device to perform a certain function or group of functions. In some embodiments, computer-executable instructions are executed on a general-purpose computer to tum the general-purpose computer into a special purpose computer implementing elements of the disclosure. The computer executable instructions may be, for example, binaries, intermediate format instructions such as assembly language, or even source code.

Those skilled in the art will appreciate that the disclosure may be practiced with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

The systems and methods described herein may utilize various communication protocols including, for example, data transmission media, communications devices, Transmission Control Protocol (“TCP”), Internet Protocol (“IP”), File Transfer Protocol (“FTP”), Telnet, Hypertext Transfer Protocol (“HTTP”), Hypertext Transfer Protocol Secure (“HTTPS”), Session Initiation Protocol (“SIP”), Simple Object Access Protocol (“SOAP”), Extensible Mark-up Language (“XML”) and variations thereof, Simple Mail Transfer Protocol (“SMTP”), Real-Time Transport Protocol (“RTP”), User Datagram Protocol (“UDP”), Global System for Mobile Communications (“GSM”) technologies, Code Division Multiple Access (“CDMA”) technologies, Time Division Multiple Access (“TDMA”) technologies, Short Message Service (“SMS”), Multimedia Message Service (“MMS”), radio frequency (“RF”) signaling technologies, Long Term Evolution (“LTE”) technologies, wireless communication technologies, in-band and out-of-band signaling technologies, and other suitable communications networks and technologies.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

The following discussion is directed to systems, methods, tools/devices, applications, and interfaces that operate utilizing a novel technique of capturing images (and/or video) and aggregating thermal and visible images to document treatment locations for cryotherapy. The inventive subject matter can be used to automatically identify the treatment location(s) of treated skin lesions or other abnormalities, which can be accurately captured with thermal imaging in a limited window of time, typically in the range of seconds (e.g., maximally, to one minute). Moreover, as image recognition is most accurate and effective for visible images, the aggregation of the thermal spectrum image with the visible image affords the important opportunity for image recognition using software and/or artificial intelligence/machine learning models.

The inventive subject matter contemplates a button, trigger, or other actuator to actuate or trigger the capture of the thermal spectrum and visible spectrum images, preferably in a synchronous fashion, i.e., simultaneously and in the requisite time frame subsequent to cryotherapy treatment. While the images could of course be captured sequentially with respect to one another, the synchronous capture of the images ensures that the same or near similar frame is captured by the respective cameras.

As such, the inventive subject matter is directed to tools for a medical provider to notate a patient's electronic medical record accurately and more efficiently with documentation of cryotherapy treatment. The tools of the present invention uniquely capture and generate composite imaging of thermal and visible spectra. The present invention further utilizes these tools in methods, applications, interfaces, and systems, as discussed herein. Moreover, these tools and related methods, application, interfaces, and systems advantageously eliminate the requirement for a secondary recording person.

Although the below discussion focuses on the capturing and analysis of images, it is contemplated that in the embodiments described below, videos could instead be captured and analyzed in place or, or in addition to, images, and the below discussion should be understood to incorporate the use of images and/or videos.

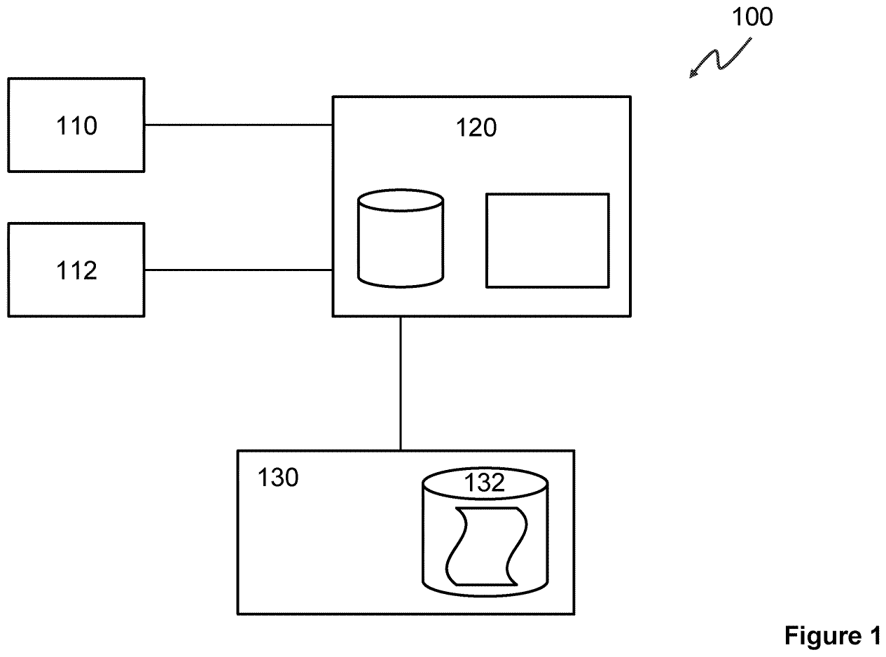

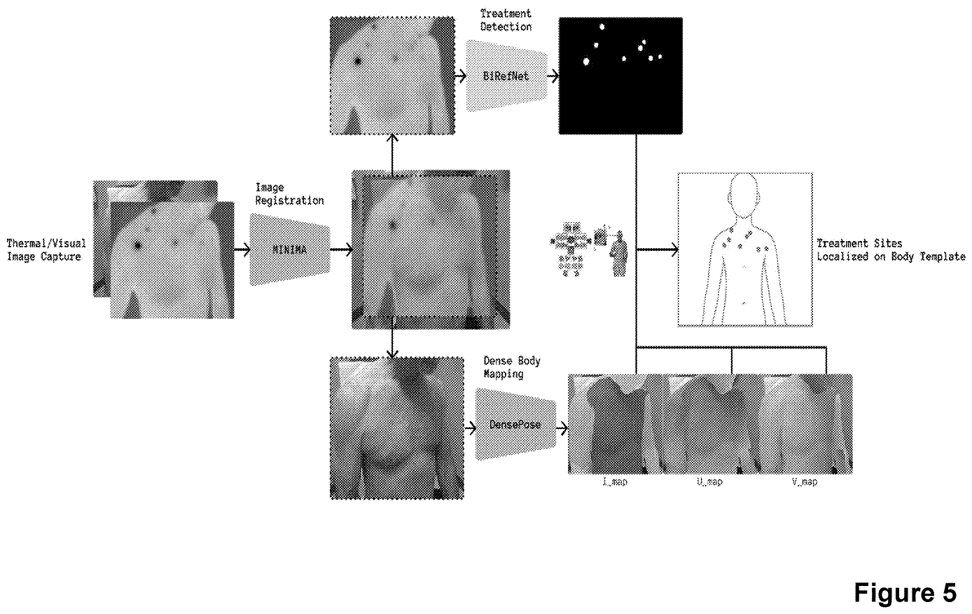

FIG. 1 illustrates one embodiment of a medical imaging and documentation system 100 for documenting cryogenic therapy of a patient. The system 100 preferably utilizes a machine learning model, which may be stored on server 120. The machine learning model processes dual-spectrum imaging data to automatically detect, localize, and map treatment sites as discussed herein and as shown in FIG. 5.

System 100 preferably comprises a mobile computing device 102 having a thermal spectrum camera 110 configured to generate a first image and a visible spectrum camera 112 configured to generate a second image. Preferably, the thermal spectrum camera 110 and the visible spectrum camera 112 are integrated with the mobile computing device 102. Of course, it is contemplated that the thermal spectrum camera 110 and the visible spectrum camera 112 could be separately housed and communicatively coupled with the mobile computing device 102. In preferred embodiments, the mobile computing device is handheld and can be mounted to a pressurized container of cryogenic fluid. However, in other embodiments, it is contemplated that the mobile computing device could comprise a wearable device such as glasses or other wearables that can incorporate one or both of the thermal spectrum camera 110 and the visible spectrum camera 112.

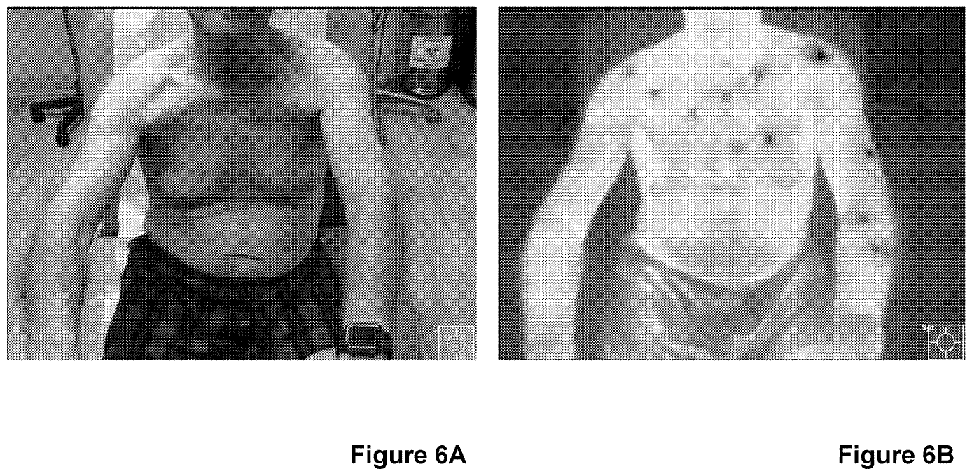

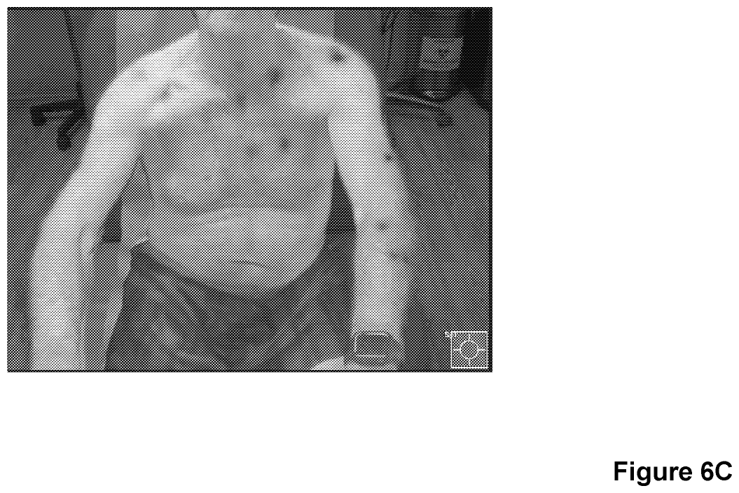

Preferably, the thermal spectrum camera 110 captures an image in the thermal spectrum while the visible spectrum camera 112 captures an image in the visible spectrum. The thermal spectrum image data may include radiometric image data representing temperature values for pixels or regions in a field of view. Preferably, the two images are captured simultaneously when actuating the trigger or actuator of the mobile computing device 102; however, in some embodiments, the images may instead be captured sequentially. FIG. 6A illustrates an example of an image captured in the visible spectrum, while FIG. 6B illustrates an example of an image captured in the thermal spectrum.

Of course, it is also contemplated that one or both of cameras 110, 112 could instead be configured to capture video recordings in their respective spectrums. In such embodiments, it is contemplated that the videos, still images from the videos, and/or related information can be captured and then transmitted and/or further analyzed.

In some embodiments, the thermal spectrum camera 110, also known as an infrared camera, captures temperatures of object surfaces, i.e., a skin lesion's surface after cryogenic therapy treatment. Preferred thermal spectrum cameras comprise an image resolution that affords diagnostic capability by the provider. In particular embodiments, the image resolution affords more advanced machine learning analysis, e.g., image recognition, alone or in the aggregate as incorporated into the composite spectrum image. The thermal spectrum image allows the ability to capture the thermal spectrum of the electromagnetic radiation emitted from a skin lesion treated by cryotherapy.

In certain embodiments, the thermal spectrum camera 110 may also produce or be calibrated to produce temperature readings that may be stored for transfer to a server or EMR, e.g., that may or may not be associated with the spectrum data being stored. In certain embodiments, the temperature readings may assist in diagnostic or characterization analysis, e.g., may be useful for distinguishing the different diagnoses.

In some embodiments, the visible spectrum camera 112 captures images (e.g., a skin lesion's surface after cryotherapy treatment) utilizing wavelengths of light from 400~700 nm, which is the same spectrum that the human eye perceives, e.g., capturing light in red, green, and blue wavelengths (RGB) for accurate color representation. Preferred visible spectrum cameras comprise an image resolution that affords diagnostic capability by the provider. In particular embodiments, the image resolution affords more advanced machine learning analysis, e.g., image recognition, alone or in the aggregate as incorporated into the composite spectrum image.

The visible spectrum image allows the ability to capture the visible spectrum of the electromagnetic radiation emitted from a skin lesion treated by cryotherapy and allows identification of treatment locations of the cryotherapy.

The system 100 further comprises a server 120 having a database, where the server is configured to receive the first image and the second image and/or information related thereto. The server 120 preferably is configured to aggregate or combine the first image with the second image to create a composite image, and then analyze the first image, the second image, and/or the composite image to identify a set of treatment locations on the patient. Alternatively, the server 120 may receive the composite image that incorporates both the first and second image. The set of treatment locations may be stored in the database.

It is contemplated that the mobile computing device 104 may store encounter data in encrypted local storage and synchronize the encounter data with the server 120 when network connectivity is available or the mobile computing device 104 is physically coupled with the server 120. The encounter data may include patient data, encounter data, visible spectrum images, thermal spectrum images, composite images, video data, audio data, temperature data, treatment site detections, lesion annotations, anatomic localization records, audit log entries, and provider notes.

Preferably, when the first and second images are aggregated or combined, a composite spectrum image is produced. Preferably, the composite spectrum image incorporates all or relevant segments of each of the received visible spectrum and thermal spectrum images. An exemplary composite spectrum image is shown in FIG. 6C. This aggregating process results from the formation or calculation by combination of the thermal spectrum image and the visible spectrum image to create the composite spectrum image.

It is contemplated that aggregating the thermal and visual images can include aligning identical points of the thermal spectrum image with those of the visible spectrum image such that the images overlap relative to the patient. In certain embodiments, the thermal spectrum image is overlaid on the visible spectrum image to align a visible patient structure to the thermal spectrum images. While the visible spectrum and thermal spectrum images are preferably captured simultaneously or in near instant succession, it is possible that slight movements during capture as well as differences in the thermal and visual camera specifications may cause some perspective distortion and misalignment between the captured images. To enable precise mapping between the visible spectrum and thermal spectrum image, the system 100 preferably utilizes a variant of the multimodal image matching (MINIMA) framework with specific optimization for thermal-visual registration to create the composite image. In particular embodiments, analysis performed on the visible spectrum image may be mapped to the thermal spectrum image and vice versa.

The analysis process by the server 120 may include using a machine learning model (treatment site detection model) that is trained to detect the treatment sites. As one example, the system 100 and server 120 preferably implements a custom-trained variant of a bidirectional refinement network (BiRefNet), which has been optimized specifically for thermal imagery analysis. Using the trained machine learning model, the server 120 can extract fine details and complex shapes from thermal data patterns, for example. The machine learning model is preferably trained on a combination of synthetically generated thermal images and annotated thermal images, which present diverse treatment patterns across various anatomical regions and patient demographics, to ensure accuracy of the machine learning model.

As shown in FIG. 5, for example, the first image, the second image, and/or the composite image can be analyzed by the server 120 to identify the set of treatment locations on the patient. As desired, the server 120 may display the visible spectrum image data, the thermal spectrum image data, or a composite representation that combines visual information with thermal information. For example, treatment locations can then be identified by detecting areas on the composite spectrum image or the thermal spectrum image that are cooler than surrounding areas. Location markers can be assigned to each of the treatment areas by analyzing the visible spectrum image, for example, and determining the coordinates of each treatment location on the patient. The server 120 preferably associates the captured data with one or more of a patient record, an encounter record, a provider identifier, a timestamp, a procedure type, a diagnosis, a body region, and/or a site annotation.

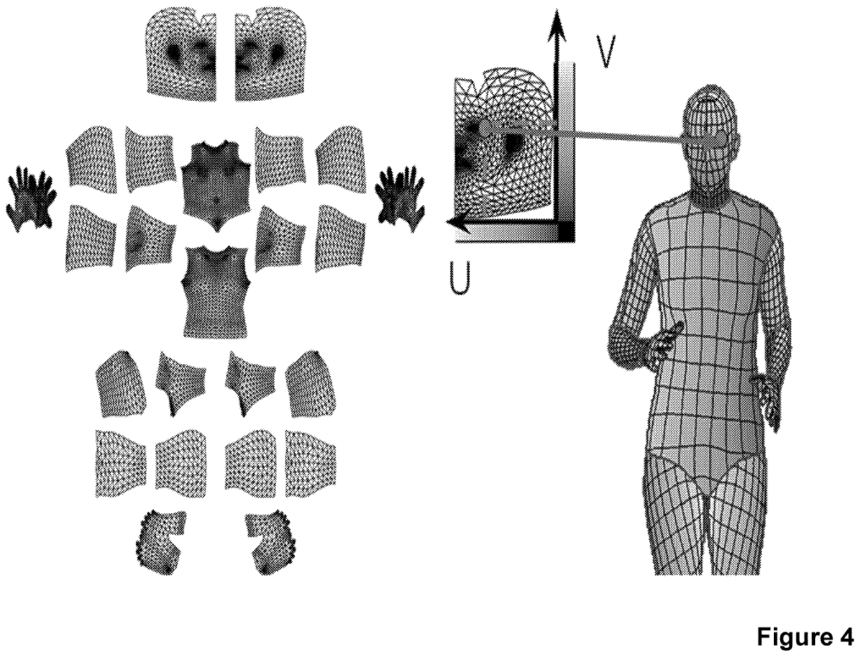

It is contemplated that treatment site localization can be determined within standardized anatomical contexts through a DensePose-based approach that generates direct surface-to-template mappings. An example of this is shown in FIG. 4. Such approach utilizes a trained model to map two-dimensional treatment site coordinates to standardized UV space. As shown in FIG. 4, the coordinates can be mapped to twenty-four distinct anatomical regions with high-resolution mapping. Such process advantageously captures partial body captures that are common in clinical settings.

As one example, a received image can be processed by the trained model to extract relevant features. Potential regions of the image having a human body can be identified. Instance segmentation can then be performed to differentiate between regions of the human body and generates a pose based on the segmentation. UV coordinates can then be predicted for each pixel within the region of interest, thereby mapping the pixel to a three-dimensional body model.

This approach utilizes a trained DensePose model, which generally determines correspondences between an RGB image and a surface-based representation of the human body, thereby mapping each pixel of the image to a specific surface part of the body. The mapped coordinates can then be determined to correspond to a part on a part of the body as shown in FIG. 4.

It is contemplated that the model can be trained using proprietary datasets and training techniques. This includes, for example, aggressive image augmentation strategies, balanced dataset selection combining close-up and full-body human images, and image perspectives representative of a clinical context. It is preferred that custom human and skin segmentation models are utilized to constrain the area of analysis to relevant treatment areas and improve performance of image matching steps.

To ensure that the capture of multiple images does not lead to redundant documentation of treatment sites, the server 120 is preferably configured to review for duplicate treatment sites before causing a patient's medical record to be notated. Because the extent of image features is limited in thermal imaging, it is contemplated that overlapping visual images are first identified before thermal images are compared to classify new or duplicate treatment sites.

In a first step, visual image matching can be used to extract robust visual features invariant to lighting and perspective changes and match corresponding regions across multiple image captures. Using keypoint matching, the server 120 can determine images with minimum required overlap for subsequent thermal image matching. Such keypoint matching may implement SuperPoint/LightGlue8 sparse keypoint matching, for example.

Next, the server 120 can be configured to use thermal image matching and employ dense correspondence for thermal image alignment to (i) establish pixel-level correspondences between thermal signatures; (ii) account for temporal decay in thermal patterns to determine primary image associated with a treatment site; and (iii) determine treatment sites that are newly captured vs duplicate captures from the previous image.

After the analysis, the server 120 preferably transmits the set of treatment locations to a medical record system 130 to document the treatment locations on the patient. In this manner, a patient's medical record can be notated or updated with the treatment locations and/or information regarding the treatment locations. This is preferably done automatically by the system 100, although some manual interaction or verification could occur, if desired.

Where location markers are assigned, it is contemplated that the server 120 transmits the location markers to the medical record system 130 to automatically document the treatment locations on the patient in the medical record of the patient. It is further contemplated that one or more of the thermal spectrum image, the visible spectrum image, and the composite spectrum image could be transferred to the medical record system 130 from the server 120.

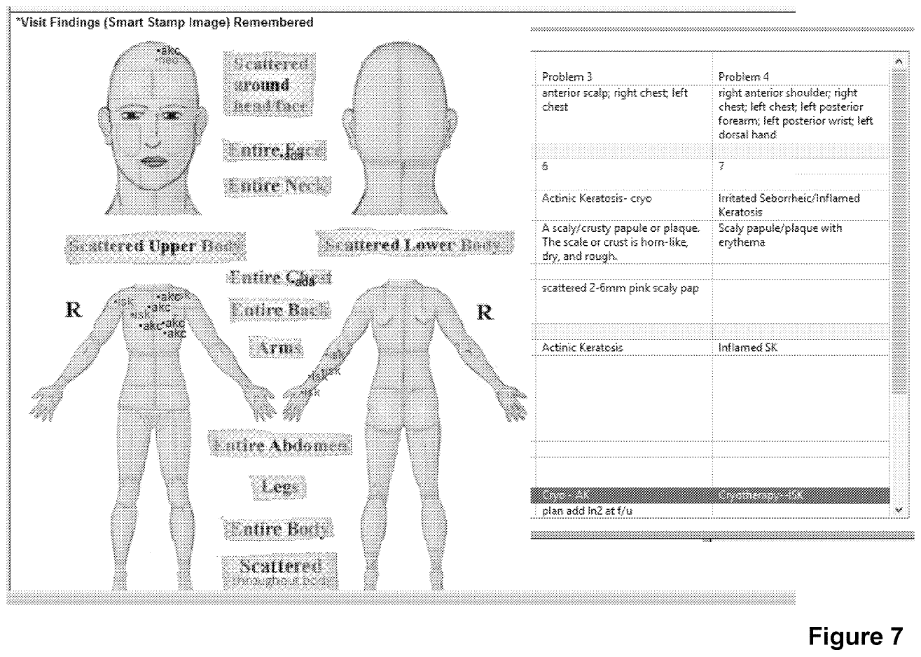

The treatment locations and/or location markers can be used to update a patient's medical record by notating the treatment locations on the medical record such as shown in FIG. 7 and/or could be saved as a pdf or other document file in the patient's file for future reference.

In some embodiments, it is contemplated that the system 100 could further comprise a medical notation API or web application configured to interface the server 120 with a database 132 of the medical record system 130. Such interface may serve as a primary tool for clinical review, documentation refinement, and EMR integration, and can provide comprehensive patient management with individual or batch profile creation, encounter documentation with synchronized thermal and visual image review, interactive body template selectors for anatomical mapping of treatment sites, visualization and annotation tools for enhanced clinical interpretation, and structured data export to common EMR systems.

In some embodiments, the application's architecture implements a stateless frontend communicating with a secure API backend (e.g., Nestjs), ensuring scalability and performance even with high-resolution imagery. Importantly, the interface additionally allows for fine-grained role-based access control for multi-provider practices and audit logging of all documentation activities.

In addition to the core components discussed above, the inventive subject matter can incorporate several image processing methods that serve to refine and validate results across the image processing pipeline. For example, a metric depth prediction model (based on the Depth Pro9 model architecture) can be used to constrain the expected size of treatment site areas based treatment area measurements derived from depth predictions.

The inventive subject matter discussed herein advantageously addresses the technical challenges unique to clinical thermal imaging, including for example, low thermal camera resolution, temporal decay characteristics of cryotherapy thermal signatures, partial body captures common in clinical dermatology, variable patient positioning between image acquisitions, and a need for precise anatomical localization despite limited visual landmarks.

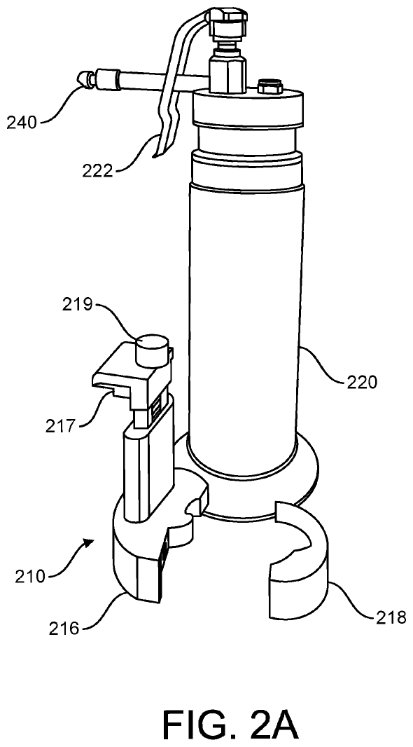

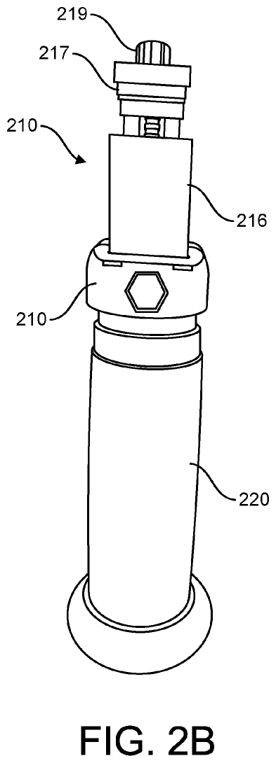

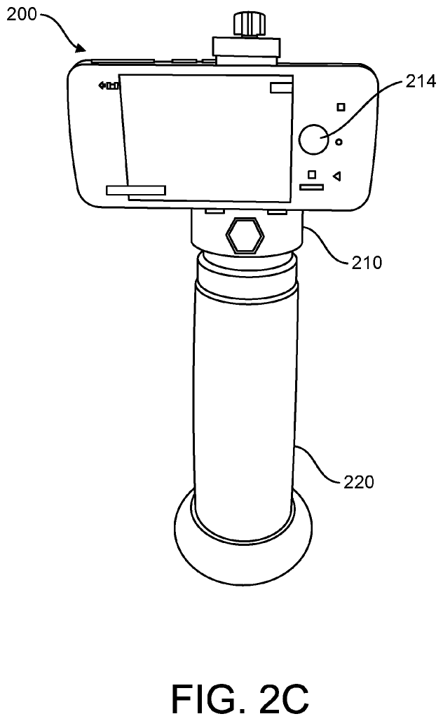

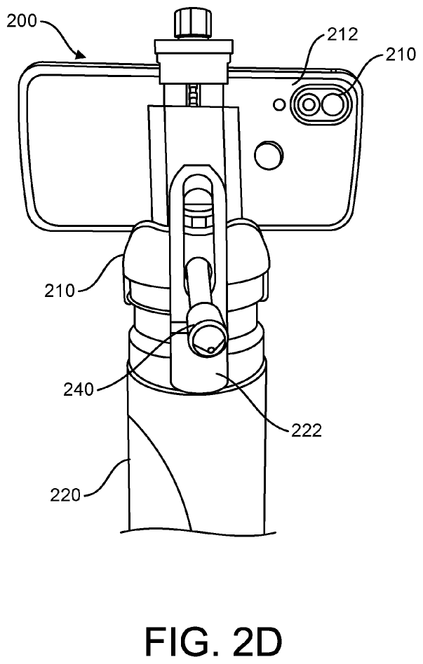

FIGS. 2A-2D illustrates various views of one embodiment of a mount 210 and an image capture device 200 that can be used with the systems and methods described herein. The image capture device 200 is preferably supported by the mount 210 configured to couple the image capture device 200 to a cryogenic fluid container 220, a cryogenic fluid delivery instrument, a handheld medical instrument, a mobile device, or a support structure near a patient. The image capture device 200 may be handheld or may be incorporated into a mobile phone, tablet, or other portable electronic device.

The image capture device 200 preferably comprises a thermal spectrum camera 210, a visible spectrum camera 212, and a trigger mechanism 214 configured to trigger the thermal spectrum camera 210 to capture the thermal spectrum in the first image and the visible spectrum camera 212 to capture the visible spectrum in the second image. While the trigger mechanism 214 preferably causes the images to be captured synchronously, it is contemplated that the images may instead by captured sequentially. The trigger mechanism 214 may be a digital button on a graphical user interface, as shown, as well as a physical button, a shutter release, or other actuator that causes the cameras to capture the images. Hands-free actuators are also contemplated including a voice-actuated trigger (i.e., actuated by voice-commands) or remotely actuated trigger, e.g., Bluetooth™ actuated or actuated via application control (e.g., through a user interface). In preferred embodiments, the image capture device 200 is communicatively coupled with a server, such as server 120.

Preferably, the image capture device 200 is mounted to a pressurized container 220 using the mount 210. The mount 210 can maintain the visible spectrum camera 212 and the thermal spectrum camera 210 in a predetermined orientation relative to a treatment area or relative to each other. As shown, one embodiment of the camera mount 210 comprises a first piece 216 and a second piece 218 that interlock to secure the camera mount 210 to the pressurized container 220. The first piece 216 and the second piece 218 can be secured to one another by any commercially suitable means, including for example, magnets, screws, claps, and other fasteners or combinations thereof, which permit the two pieces to be removably attached to one another. This allows the camera mount 210 to be moved to a different pressurized container when the first container is empty for example.

It is preferred that the pressurized container 220 is configured to hold a cryogenic fluid. The container 220 has an actuator 222 that when engaged causes a valve to open to release some of the cryogenic fluid from the container 220 via a cryogenic fluid applicator 240. In particular embodiments, the cryogenic fluid applicator 240 is adapted to dispense the cryogenic fluid to skin lesions or other abnormalities of the patient. In specific embodiments, the cryogenic fluid applicator 240 is a BRYMILL™ cryogenic fluid applicator, e.g., a BRYMILL™ Cry-AC or Cry-AC 3.

Though not required, in some embodiments, it is contemplated that the actuator 222 can be configured to automatically cause the thermal spectrum camera 210 to capture the first image and the visible spectrum camera 212 to capture the second image at a preset time after some of the cryogenic fluid is expelled from the pressurized container 220 due to actuation of the actuator 222. In other words, the actuator 222 serves to actuate or initiate the capture of thermal and visible spectra, preferably in a synchronous manner. In particular embodiments, the preset time may range from 1-2 seconds, from about 1 to 60 seconds, from 2-30 seconds, and more preferably within 15 seconds, after application of the cryogenic fluid.

In other embodiments, it is contemplated that the cameras 210, 212 are manually actuated after the cryogenic fluid is expelled from the pressurized container 220, such as by using trigger mechanism 214.

As discussed above, the thermal spectrum image and the visible spectrum image are aggregated to create the composite spectrum image that can be stored on a machine-readable medium. It is contemplated that the machine-readable medium is a network server disk or a cloud storage, for example. The image capture device 200 may comprise the machine-readable medium. Alternatively, the images can be transmitted to a server which can comprise a machine-readable medium.

In certain embodiments, the aggregation of the thermal spectrum image and the visible spectrum image may be performed within the image capture device 200 rather than the server.

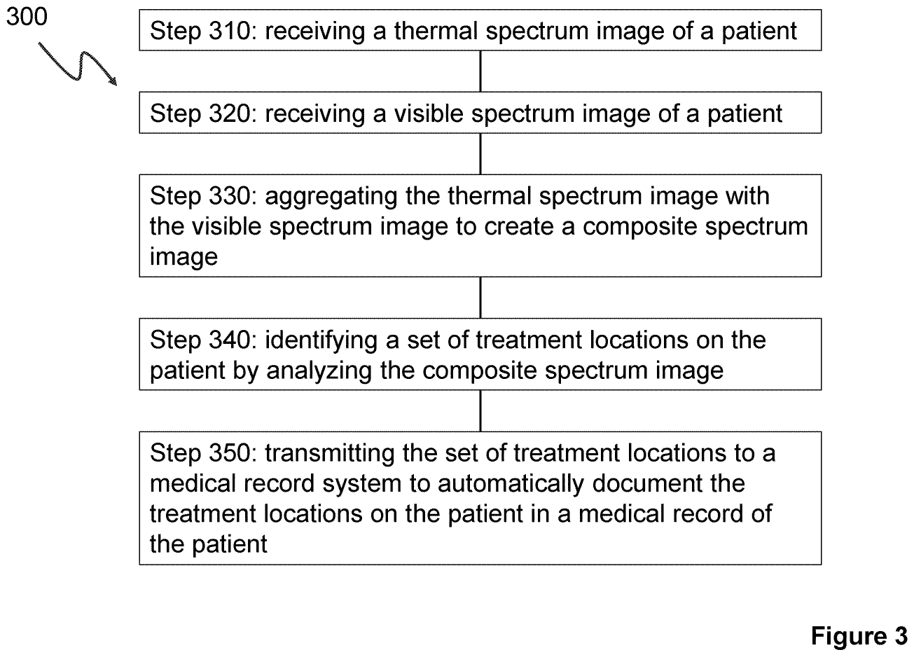

FIG. 3 illustrates one embodiment of a method 300 of documenting cryogenic therapy of a patient. The method 300 comprises receiving a thermal spectrum image of a patient in step 310 and receiving a visible spectrum image of the patient in step 320. These images may be received simultaneously or sequentially by a server or other device.

Preferably, the thermal spectrum image and the visible spectrum image of the patient were taken synchronously, though sequentially captured images are also contemplated. In such embodiments, it is contemplated that a thermal spectrum camera is used to capture the thermal spectrum image and a visible spectrum camera is used to capture the visible spectrum image, with the cameras triggered using a manual or digital button or other actuator. Preferably, the step of capturing the images occurs a preset time after dispensing of a cryogenic fluid on the patient and can occur automatically or manually by the provider, for example.

The thermal spectrum image is aggregated with the visible spectrum image to create a composite spectrum image in step 330. In certain embodiments, the step of aggregating comprises aligning identical points of the thermal spectrum image with those of the visible spectrum image such as discussed above and so that the images overlap relative to the patient.

A set of treatment locations on the patient is identified in step 340 by analyzing the thermal spectrum image, the visible spectrum image, and/or the composite spectrum image. This could include, for example, detecting areas on the thermal spectrum image that are cooler than surrounding areas. The anatomic locations of those areas can each be assigned a location marker based on its location relative to the patient through an analysis of the visible spectrum image, for example. Preferably, this step 340 utilizes the techniques described herein and shown in FIGS. 4 and 5.

After identification, the set of treatment locations are transmitted to a medical record system in step 350 to automatically document the treatment locations on the patient in a medical record of the patient. Where location markers are assigned, it is contemplated that the location markers can be transmitted to the medical record system to automatically document the treatment locations on the patient in the medical record of the patient.

In some embodiments, one or more of the thermal spectrum image, the visible spectrum image, and the composite spectrum image can be transferred to the medical record system.

In another aspect, an application is contemplated comprising a non-transitory machine-readable medium having instructions stored thereon for execution by a processor to perform the method described above or a method comprising the following steps. A thermal spectrum image captured by a thermal spectrum camera is stored on a server. A visible spectrum image captured by a visible spectrum camera is also stored on the server.

The thermal spectrum image and the visible spectrum image are aggregated using a processor of the server to create a composite spectrum image. This can occur by aligning identical points of the thermal spectrum image with those of the visible spectrum image such that the overlapping images create the composite spectrum image.

Areas are detected on the composite spectrum image that are cooler than surrounding areas using the processor. Each of the areas are assigned a location marker based on its location relative to the patient using the processor. This may be done, for example, by an analysis of the visible spectrum to determine anatomic locations.

The server interfaces with a medical record database of a medical record system and transfers the location markers to the medical record database of the EMR system, such that the medical record of the patient is automatically notated with the treatment locations.

In some embodiments, and as discussed above, the steps can include synchronously or sequentially capturing the thermal spectrum image using the thermal spectrum camera and capturing the visible spectrum image using the visible spectrum camera a set time after cryogenic fluid is dispensed.

Another embodiment of a method for capturing cryogenic therapy (e.g., of skin lesions) into a patient's EMR is described below. The method comprises capturing the visible and thermal spectrum images using visible and thermal spectrum cameras and aggregating these images to create a composite spectrum image.

A first step comprises causing a thermal spectrum camera to capture a thermal spectrum image and a visible spectrum camera to capture a visible spectrum image. The thermal spectrum image and the visible spectrum image can be stored on a non-transitory machine-readable medium. This preferably occurs after dispensing a cryogenic fluid, e.g., liquid nitrogen, to a patient.

In certain embodiments, dispensing the cryogenic fluid triggers the capture of the thermal spectrum and the visible spectrum images. In such embodiments, the visible spectrum and thermal spectrum images are captured after a minimal delay subsequent to application of the cryogenic fluid. In particular embodiments, the minimal delay may range from about 1 to 60 seconds (e.g., from about 2-30 seconds, e.g., from about 2-30 seconds, e.g., less than about 15 seconds) after application of the cryogenic fluid. In other embodiments, the images may be captured after manual actuation of the cameras.

The thermal spectrum image and the visible spectrum image can be aggregated to create a composite spectrum image, which may be stored on the machine-readable medium or other location.

An interface can be created or opened with an EMR database of an EMR system. The composite spectrum image can be transferred to the EMR database of the EMR system, such that the composite spectrum image is medically notated in the EMR database of the EMR system. In certain embodiments, the method further comprises transferring the thermal spectrum image and the visible spectrum image to the EMR database of the EMR system.

As discussed above, the systems and methods of the present invention may comprise the step of dispensing a cryogenic fluid, e.g., liquid nitrogen, to a patient. The purpose of dispensing the cryogenic fluid is to freeze the patient's skin quickly and then allow it to slowly thaw to cause maximum destruction to targeted skin cells. In certain embodiments, the present invention applies a cryogenic fluid using a cryogenic fluid applicator, e.g., a spray device, such as discussed above. In certain embodiments, the cryogenic fluid is liquid nitrogen. In particular embodiments, the cryogenic applicator is adapted to dispense the cryogenic fluid to skin lesions of a patient.

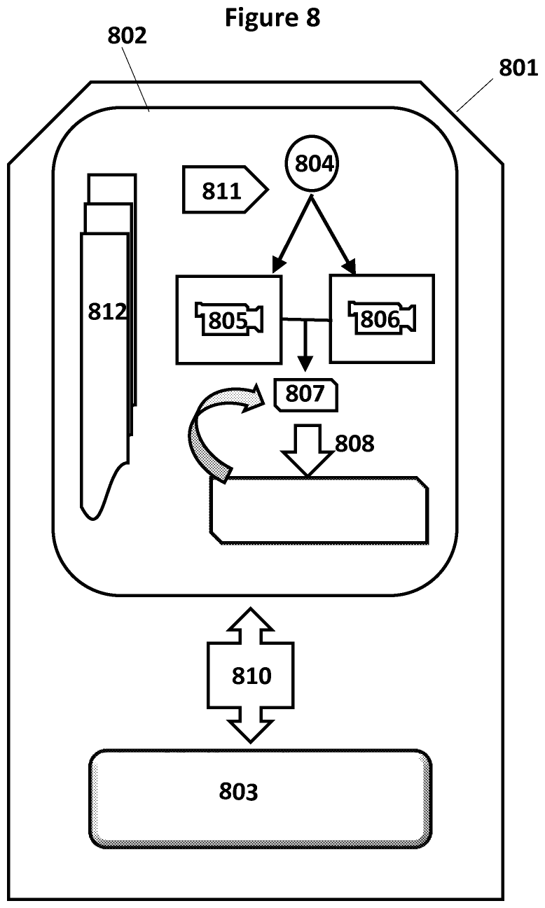

FIG. 8 illustrates a schematic view of another embodiment of a system for medical notation of the present invention. System 801 comprises composite spectrum image capture tool 802 and EMR database 803 of an EMR system. Composite spectrum image capture tool 802 comprises actuator 804, with limited manual interface that synchronously triggers the thermal spectrum camera 805 to capture the thermal spectrum image and the visible spectrum camera 806 to capture the visible spectrum image. The thermal spectrum and the visible spectrum images are stored on the same machine-readable medium 807, and then aggregated 808 to create a composite spectrum image 809 that is stored on machine-readable medium 807.

The system 801 further comprises medical notation API 810 that interfaces the composite spectrum image capture tool 802 and EMR database 803 of the EMR system.

The composite spectrum image capture tool 802 further comprises a cryogenic fluid applicator 811.

The composite spectrum image capture tool 802 further comprises user interface 812.

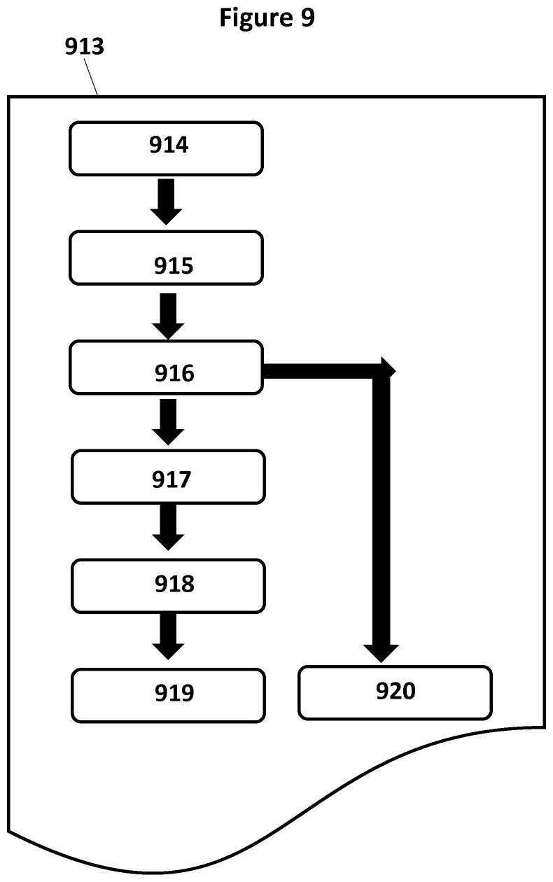

FIG. 9 illustrates a schematic view of another embodiment of a method 913 of capturing cryogenic therapy that may be implemented as an application, which in turn may operate in the composite spectrum image capture tool 802 of FIG. 8.

Method 913 of capturing cryogenic therapy (e.g., of skin lesions) into a patient's EMR comprises the step of dispensing 914 a cryogenic fluid, e.g., liquid nitrogen, to a patient. The method 912 further comprises the step of synchronously triggering 915 a thermal spectrum camera to capture a thermal spectrum image and a visible spectrum camera to capture a visible spectrum image. The thermal and visible spectrum images are stored 916 on a machine-readable medium. The stored thermal and visible spectrum images are aggregated 917 to create a composite spectrum image, e.g., that is stored on the machine-readable medium. Interfacing 918 with an EMR database of an EMR system allows for transferring 919 of the composite spectrum image (e.g., that is stored on the machine-readable medium) to the EMR database of the EMR system, such that the composite spectrum image is medically notated in the EMR database of the EMR system.

The method 913 may further comprise the step of transferring 920 the thermal spectrum image and the visible spectrum image to the EMR database of the EMR system.

In certain embodiments, the thermal spectrum camera may also produce or be calibrated to produce temperature readings that may be stored for transfer to the EMR, e.g., that may or may not be associated with the spectrum data being stored. In particular embodiments, the temperature readings may assist in diagnostic or characterization analysis, e.g., may be useful for distinguishing the different diagnoses.

It is contemplated that the machine-readable medium may be located locally, such as physically interfaced with one or both of the thermal and visible spectrum cameras collecting the thermal and visible spectrum, or otherwise disposed on the image capture device described above. The machine-readable medium may also be a network server disk, wirelessly interfacing with the thermal and visible spectrum cameras and/or the image capture device, collecting the thermal and visible spectrum, e.g., using an app network server disk. In still other embodiments, the systems and methods described herein may avoid the step of storing the thermal and visible spectrum image by directly aggregating the images upon capture.

In certain embodiments, the interfacing with the medical record system may be bi-directional. In other embodiments, the interfacing may be unidirectional. In particular embodiments, such interfacing may be achieved through a graphical user interface. In one aspect, the interfacing is between an EMR database of an EMR system and an application implementing the methods of the present invention (e.g., which serves also to capture visible and thermal spectrum images from cameras, store them, aggregate them, and transfer them to an EMR database). In certain embodiments, the interfacing is accomplished through an API as discussed above.

Still further, it is contemplated that the image capture device further comprises a user interface. The user interface may be located on the machine-readable medium. In certain alternative embodiments, the user interface may be located on a separate device, e.g., a tablet computing device. In some embodiments, the image capture device may be wearable by the provider, for example. In such embodiments, the tool may be configured as part of eyewear, e.g., glasses adaptable to left or right eye dominant users. In certain embodiments, the trigger mechanism is hardware and processors that are configured for voice-activation triggering, augmented reality triggering, laser alignment triggering.

In particular embodiments, the thermal spectrum camera comprises a processor to facilitate the interaction with the trigger. In certain embodiments of the present invention, the thermal spectrum camera may also comprise a beam or laser projection component, e.g., to allow provider to center the captured image. In certain embodiments, the thermal spectrum camera is a Flir Lepton thermal camera (e.g., Flir One Edge Pro, Real Wear Navigator 520, and Arsenz Thermoglass).

In particular embodiments, the visible spectrum camera comprises a processor to facilitate the interaction with the trigger. In certain embodiments of the present invention, the visible spectrum camera may also comprise a beam projection component, e.g., to allow provider to center the captured image. In certain embodiments, the visible spectrum camera utilizes a CMOS 160×120 pixel sensor.

The inventive subject matter discussed above advantageously affords a medical provider the ability to perform cryogenic treatment, e.g., liquid nitrogen treatment, and concordantly chart the procedure within an electronic medical record (EMR) database without the need for a separate scribe, for example. The inventive subject matter allows the provider to both initiate and complete the procedure, while simultaneously capturing the visible and thermal spectrum images of the treated spots into the patient's medical record. The visible and thermal spectrum images are captured by visible and thermal spectrum cameras and are aggregated to create a composite spectrum image.

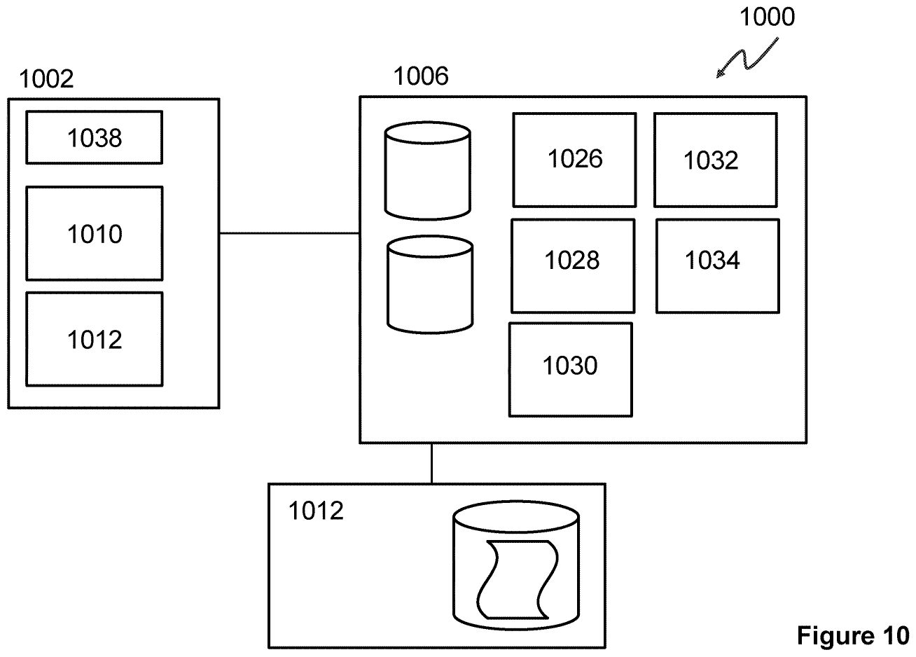

FIG. 10 illustrates another embodiment of a medical imaging and documentation system 1000. The system 1000 preferably utilizes a machine learning model, which may be stored on a server 1006. The machine learning model may process dual-spectrum imaging data to automatically detect, localize, and map treatment sites as discussed herein and as shown in FIG. 5.

System 1000 preferably comprises a mobile computing device 1002 having a thermal spectrum camera 1010 configured to generate a first image and a visible spectrum camera 1012 configured to generate a second image. Preferably, the thermal spectrum camera 1010 and the visible spectrum camera 1012 are integrated with the mobile computing device 1002. Of course, it is contemplated that the thermal spectrum camera 1010 and the visible spectrum camera 1012 could be separately housed and communicatively coupled with the mobile computing device 1002. In preferred embodiments, the mobile computing device 1002 is handheld and can be mounted to a pressurized container of cryogenic fluid. However, in other embodiments, it is contemplated that the mobile computing device could comprise a wearable device such as glasses or other wearables that can incorporate one or both of the thermal spectrum camera 1010 and the visible spectrum camera 1012.

Preferably, the thermal spectrum camera 1010 captures an image in the thermal spectrum while the visible spectrum camera 1012 captures an image in the visible spectrum. The thermal spectrum image data may include radiometric image data representing temperature values for pixels or regions in a field of view. Preferably, the two images are captured simultaneously when actuating the trigger or actuator of the mobile computing device 1002; however, in some embodiments, the images may instead be captured sequentially. FIG. 6A illustrates an example of an image captured in the visible spectrum, while FIG. 6B illustrates an example of an image captured in the thermal spectrum.

The server 1006 comprises software configured to process the images (or data related to the images) received from the mobile computing device 1002. The server 1006 may include a site detection module 1026, an image registration module 1028, an anatomic mapping module 1030, a duplicate resolution module 1032, and a site re-identification module 1034. Although these modules are described separately, the functions of two or more modules may be combined, distributed across multiple servers or computing devices, or performed in different orders.

The site detection module 1026 is configured to identify a treatment site, biopsy site, lesion site, skin condition site, or other site of interest in image data, such as by using the above-described methods. In some embodiments, the site detection module 1026 identifies a cryotherapy treatment site based on a thermal signature produced by application of liquid nitrogen or another cryogenic fluid to skin of the patient. The thermal signature may include a temperature value, a temperature gradient, a temperature difference relative to surrounding tissue, a cooled region size, and other information concerning the temperature of the site. The site detection module 1026 may use a trained neural network to detect cryotherapy treatment site(s).

In some embodiments, the site detection module 1026 uses a neural network model trained to identify cooled tissue regions in thermal image data. The model may be trained using annotated thermal images, synthetic thermal images, augmented images, images representing multiple body regions, images representing multiple patient positions, or images representing multiple treatment conditions. The site detection module 1026 may output a coordinate point, a bounding region, or other information associated with each detected site.

The image registration module 1028 aligns visible spectrum image data and thermal spectrum image data. In some embodiments, the visible spectrum camera 1012 and the thermal spectrum camera 1010 capture corresponding images in near temporal proximity, but the images may have slight differences in perspective, resolution, and so forth. The image registration module 1028 determines a relationship between the visible spectrum image data and the thermal spectrum image data using feature matching, camera calibration data, learned registration, or other methods and combinations thereof. The relationship may be used to transfer a detected thermal site to a visible spectrum image or to transfer a visible site annotation to a thermal spectrum image, for example.

The anatomic mapping module 1030 maps a site of interest on a patient to an anatomic coordinate space. The anatomic coordinate space may include a plurality of predefined body regions and a coordinate representation for one or more body regions, as described above. In some embodiments, the coordinate representation includes a surface parameterization having a first coordinate and a second coordinate corresponding to a surface of a three-dimensional body model. The surface parameterization may be a XY coordinate system in which X and Y coordinates represent positions on a flattened representation of a three-dimensional body surface. The anatomic mapping module 1030 may output a body region identifier and one or more coordinate values corresponding to the site of interest.

In some embodiments, the anatomic mapping module 1030 uses a dense body mapping model to map image pixels to the anatomic coordinate space. The dense body mapping model may identify a body region visible in an image, determine surface coordinates for pixels in the body region, and associate a site of interest with the corresponding body region and surface coordinates. The dense body mapping model may support partial body captures in which only a portion of a patient is visible. The dense body mapping model may be trained or configured using full-body images, close-up images, clinical-perspective images, augmented images, or combinations thereof.

It is contemplated that the anatomic mapping module 1030 may use depth information to improve coordinate assignment. The depth information may be measured by a depth sensor or estimated from one or more images. The depth information may be used to estimate camera pose, constrain an expected physical size of a treatment site, project an image coordinate to a three-dimensional body mesh, triangulate a coordinate from multiple views, or validate a site detection. The anatomic mapping module 1030 may also use human segmentation or skin segmentation to constrain analysis to body regions relevant to treatment, lesion documentation, or skin condition documentation.

The database of the server may store an anatomic localization record for each site of interest. The anatomic localization record may include a patient identifier, encounter identifier, image identifier, procedure type, diagnosis, body region identifier, surface coordinates, pixel coordinates, temperature measurements, provider annotations, timestamps, and/or audit information. The anatomic localization record may be stored independently of a particular image so that the location can be mapped to a later image, a body template, a current camera view, or a patient-specific surface representation, for example.

The duplicate resolution module 1032 can be used to identify whether a site captured in one image duplicates a site captured in another image. During a procedure, a provider may capture multiple images that partially overlap or that include the same treatment site from different perspectives. The duplicate resolution module 1032 may first identify overlapping visible spectrum images using sparse keypoint matching, feature descriptors, interest point detection, image similarity, camera pose estimation, or another visual matching techniques. After identifying candidate overlapping images, the duplicate resolution module 1032 may compare thermal images or thermal site detections to determine whether detected treatment sites correspond to the same physical site. The duplicate resolution module 1032 may select a representative image for a treatment site, suppress duplicate entries, merge measurements, or retain multiple observations in a record.

The site re-identification module 1034 can be used to re-identify a previously documented site in a later image or guide a provider to identify the previously documented site on the patient in a later encounter. In some embodiments, the site re-identification module 1034 receives a source image depicting a source region corresponding to a site of interest and a target image captured during a later encounter. The source image may be a prior procedure photograph, a biopsy photograph, a lesion photograph, a thermal image, a composite image, or an uploaded image. The target image may be captured before a surgical procedure, topical treatment, cryotherapy procedure, biopsy, follow-up examination, or monitoring encounter. The visible images can be captured using any device having a image capture module or camera, including for example, tablet PCs, smart phones, and so forth. The site re-identification module 1034 determines correspondences between the source image and the target image and transfers the source region to the target image based on the correspondences.

It is further contemplated that the site re-identification module 1034 can communicate with the mobile computing device 1002 to allow a provider to determine a site of interest on the patient during the later encounter. In other words, captured data from a prior encounter can be used to identify points of interest on the same patient in a future encounter and/or could be used to ensure surgical procedures occur at the precise location of the site of interest. This could occur by overlaying information on the mobile computing device 1002 or using that device or other another to project light or a laser to pinpoint the site of interest on the patient based on the previously captured and analyzed data. The site re-identification module 1034 may determine correspondences using coarse matching, refinement, dense image feature matching, multimodal matching, triangulation from multiple views, stored transformation parameters, stored surface coordinates, or combinations thereof.

In one example, the site re-identification module 1034 can be used to confirm an original biopsy site location when a patient presents for surgical removal of a malignant or precancerous lesion. A provider may capture or upload a source image associated with an original biopsy. The provider may draw, select, or confirm a source mask over the biopsy site. Before surgery, the provider captures a target image of the patient. The site re-identification module 1034 transfers the source mask to the target image and displays a target contour or marker corresponding to the original biopsy site. This can assist the provider in confirming the surgical location even when the biopsy site has healed or when visible landmarks have changed, for example.

Thus, it is contemplated that the system 1000 may utilize stored anatomic coordinates to guide a future procedure. This could include utilizing dense matching where two or more images are aligned pixel by pixel. However, the process is capable of comparing more than the two or more images. The provider may select a prior treatment site, lesion site, biopsy site, or condition site. The system 1000 retrieves the anatomic localization record for the selected site and maps the stored anatomic coordinates to a current image, body template, camera view, patient-specific body representation, or on the patient themself. A display may present guidance information indicating the location of the selected site. The guidance information may include a point marker, contour, highlighted body region, directional cue, distance indicator, confidence indicator, comparison view, thermal overlay, visible overlay, or instruction to reposition the image capture device 1002.

In some embodiments, the system 1000 is configured to assign an anatomic location to a new skin lesion or other condition. The provider captures an image of a lesion or condition, and the anatomic mapping module 1030 maps the lesion or condition to the anatomic coordinate space. The system 1000 stores an anatomic localization record for the lesion or condition. During a later encounter, the system 1000 may determine whether a lesion or condition in a later image corresponds to the stored lesion or condition, corresponds to another stored lesion or condition, or represents a new lesion or condition. The system 1000 may generate a record including images, measurements, color information, thermal information, size changes, diagnostic information, treatment history, and provider notes associated with the stored coordinates.

In some embodiments, the system 1000 uses thermal responsiveness to characterize a lesion, treatment site, or skin condition. Thermal responsiveness may include a measured temperature, a change in temperature over time, a cooling rate, a warming rate, a thermal contrast relative to adjacent tissue, a duration below a threshold temperature, a minimum temperature, a maximum temperature, or a temporal decay pattern. The system 1000 may store thermal responsiveness data with the anatomic localization record. The thermal responsiveness data may be used for documentation, quality review, treatment planning, follow-up comparison, or diagnostic support.

The captured images may provide real-time temperature information during a procedure. The thermal spectrum camera 1010 may provide radiometric data over a temperature range suitable for detecting cooled tissue produced by cryotherapy. The captured information may include a temperature for a selected point or a region of interest. The mobile computing device 1002 may determine that a threshold temperature has been reached, generate a notification, start a timer, stop a timer, or automatically capture an image or video frame when the threshold temperature is reached.

In some embodiments, the system 1000 uses audio data to assist image capture or documentation. A microphone 1038 may capture audio during a procedure. This can be sent to the server 1006 or the mobile computing device 1002 may detect a sound associated with discharge of cryogenic fluid and use the detected sound to initiate image capture, start a treatment timer, identify a treatment event, associate an image with a treatment site, or prompt the provider to capture an image. The mobile computing device 1002 may also receive a voice command to capture an image, start or stop video capture, annotate a site, select a diagnosis, select a procedure type, save an encounter, or mark an image for review.

In some embodiments, the mobile computing device 1002 captures video data during treatment. The mobile computing device 1002 or server 1006 may analyze a sequence of frames to determine treatment timing, freezing duration, cooling rate, warming rate, expansion of a cooled region, temperature threshold duration, or other parameters. The mobile computing device 1002 or server 1006 may select a representative frame, generate a composite image, or associate multiple frames with one anatomic localization record.

The review application may display synchronized visible spectrum and thermal spectrum images for a patient encounter. The review application may allow a provider to edit or confirm automatically detected sites, add or remove contours, assign diagnoses, select body regions, confirm re-identified biopsy sites, approve anatomic localization records, and export documentation to the electronic medical record system 1012. The review application may generate a report identifying treated sites, lesion sites, biopsy sites, diagnoses, procedure parameters, temperatures, anatomic coordinates, and provider annotations.

The modules and methods described above may be implemented using one or more processors executing instructions stored in one or more non-transitory computer-readable media. The one or more processors may be included in a mobile computing device, a server, a workstation, a cloud computing environment, an image capture device, or a combination thereof. The system 1000 may use local processing, remote processing, or distributed processing.

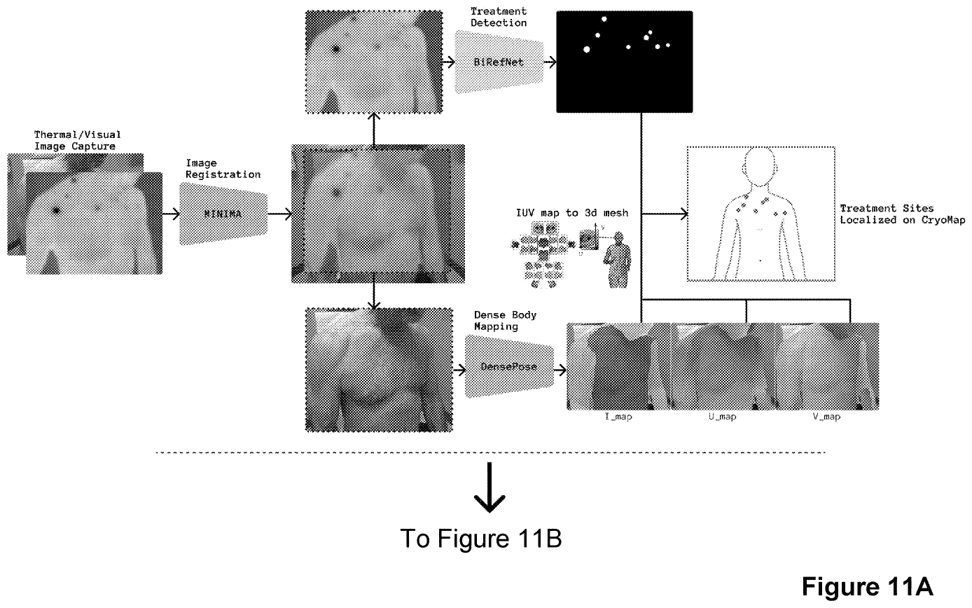

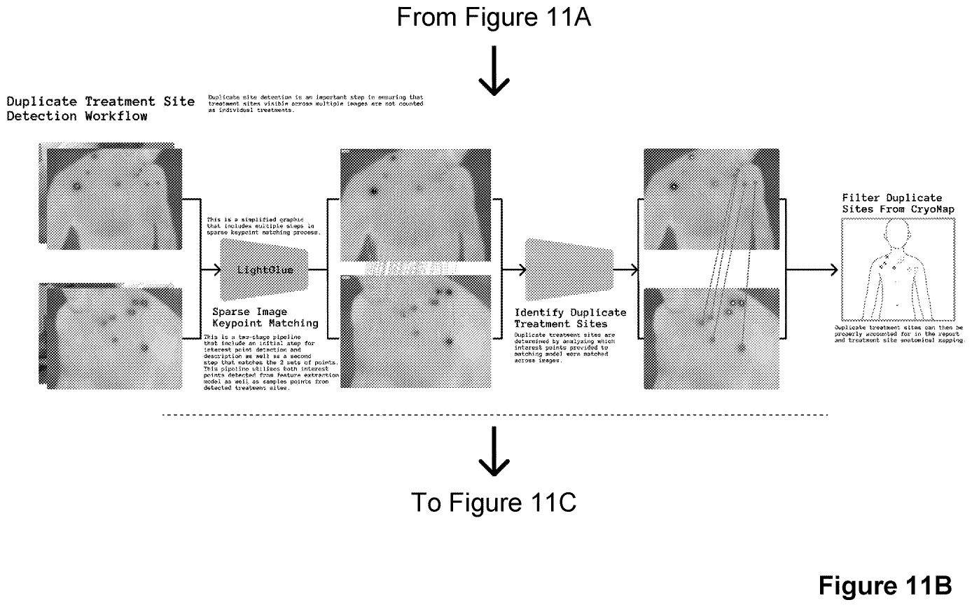

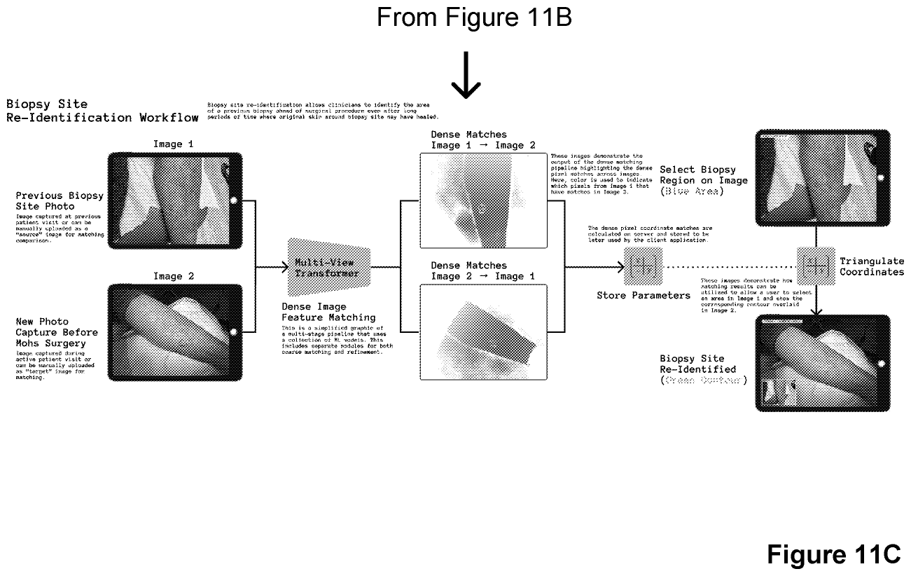

FIGS. 11A-11C illustrate an embodiment of an example workflow for mapping a detected site to an anatomic coordinate space, detecting duplicate treatment sites, and re-identifying site locations.

As shown in FIG. 11A, and as described above, thermal and visual spectrum images can be captured of a treatment area or site of interest on a patient's body. The images can be mapped to one another to create a composite image. For precise mapping between the visible spectrum and thermal spectrum images, a variant of the multimodal image matching (MINIMA) framework can be used with specific optimization for thermal-visual registration.

The composite image and/or thermal image can be analyzed by a server to detect the treatment sites. This can be done by comparing temperatures across the skin as shown in the thermal or composite images. The analysis may include using a machine learning model (treatment site detection model) that is trained to detect the treatment sites. As one example, a custom-trained variant of a bidirectional refinement network (BiRefNet) could be used, which has been optimized specifically for thermal imagery analysis. For example, treatment locations can then be identified by detecting areas on the composite image or the thermal image that are cooler than surrounding areas. Location markers can be assigned to each of the treatment areas by analyzing the visible spectrum image, for example, and determining the coordinates of each treatment location on the patient. The server 120 preferably associates the captured data with one or more of a patient record, an encounter record, a provider identifier, a timestamp, a procedure type, a diagnosis, a body region, and/or a site annotation. It is contemplated that treatment site localization can be determined within standardized anatomical contexts through a DensePose-based approach that generates direct surface-to-template mappings, such as discussed above.

As shown in FIG. 11B, and as described above, the server can also be configured to detect for duplicate treatment sites that may be visible across multiple images. This preferably occurs before a patient's medical record is updated/notates to prevent redundant documentation of treatment sites. It is contemplated that visual image matching can be used to extract robust visual features invariant to lighting and perspective changes and match corresponding regions across multiple image captures. Using keypoint matching, images having a minimum required overlap can be determined for subsequent thermal image matching. Such keypoint matching may implement SuperPoint/LightGlue8 sparse keypoint matching, for example.

Next, the server can use thermal image matching and employ dense correspondence for thermal image alignment to (i) establish pixel-level correspondences between thermal signatures; (ii) account for temporal decay in thermal patterns to determine primary image associated with a treatment site; and (iii) determine treatment sites that are newly captured vs duplicate captures from the previous image. After the analysis, duplicate sites are preferably filtered out and the remaining treatment sites can be documented. In this manner, a patient's medical record can be notated or updated with the treatment locations and/or information regarding the treatment locations.

As shown in FIG. 11C, and as described above, the server can use stored data to re-identify a previously documented site in a later image or guide a provider to identify the previously documented site on the patient in a later encounter. This could be used, for example, by clinicians who need to identify the area of a previous biopsy ahead of a surgical procedure, especially where the site may have healed and cannot be visually distinguished.

To re-identify the site, it is contemplated that a previous image stored in a databased or uploaded to the server can be used to determine the previously documented site. The previous image may be a prior procedure photograph, a biopsy photograph, a lesion photograph, a thermal image, a composite image, or an uploaded image. A new image can be captured of the region of interest on the patient and transmitted to the server. Correspondences can be determined between the previous image and the new image and transfers the previous region to the new image based on the correspondences. One or more trained models can be used. It is contemplated that Dense Image Feature Matching can be used to determine the correspondences and triangulate coordinates between the two images. Correspondences could also be determined using coarse matching, refinement, multimodal matching, triangulation from multiple views, stored transformation parameters, stored surface coordinates, or combinations thereof.

As shown, the site of interest can be identified on the new image. It is also contemplated that the server can communicate with a device to allow a provider to determine a location of the site of interest directly on the patient during a later encounter. This could occur by overlaying information on the device itself or using that device or other another to project light or a laser to pinpoint the site of interest on the patient based on the previously captured and analyzed data. Thus, in this manner, stored anatomic coordinates can be utilized to determine a location for a future procedure, for example.

Although embodiments are described in connection with cryotherapy and dermatology, the disclosed anatomic localization and future guidance techniques may be used for other medical procedures in which a site on a body surface is documented, mapped, monitored, or revisited. For example, the inventive subject matter could be used to map skin lesions that have not undergone cryotherapy. The site may be a treatment site, a biopsy site, a lesion site, a wound site, a rash site, an injection site, a topical treatment site, or another site of clinical interest.

As used herein, the language “application programming interface” or “API” are art-recognized, and used interchangeably, to describe a type of software interface, offering a service to other pieces of software, i.e., a way for two or more computer programs to communicate with each other. In contrast to a user interface, which connects a computer to a person, an application programming interface connects computers or pieces of software to each other. It is not intended to be used directly by a person (the end user) other than a computer programmer who is incorporating it into the software. An API is often made up of different parts which act as tools or services that are available to the programmer. A program or a programmer that uses one of these parts is said to call that portion of the API. The calls that make up the API are also known as subroutines, methods, requests, or endpoints. An API specification defines these calls, meaning that it explains how to use or implement them.

The term “interfacing” is art-recognized and is used herein to describe the means of communication between two entities, for example a system/tool and user data entry. In certain embodiments, the interfacing may be bi-directional. In other embodiments, the interfacing may be unidirectional. In particular embodiments, such interfacing may be achieved through a graphical user interface.

The language “machine-readable medium” is art-recognized and describes a medium capable of storing data in a format readable by a mechanical device (rather than by a human). Examples of machine-readable media include magnetic media such as magnetic disks, cards, tapes, and drums, punched cards and paper tapes, optical disks, barcodes, magnetic ink characters, and solid-state devices such as flash-based, SSD, etc. Machine-readable medium of the present invention is non-transitory, and therefore do not include signals per se, i.e., are directed only to hardware storage medium. Common machine-readable technologies include magnetic recording, processing waveforms, and barcodes. In particular embodiments, the machine-readable device is a solid-state device. Optical character recognition (OCR) can be used to enable machines to read information available to humans. Any information retrievable by any form of energy can be machine-readable. Moreover, any data stored on a machine-readable medium may be transferred by streaming over a network. In a particular embodiment, the machine-readable medium is a network server disk, e.g., an internet server disk, e.g., a disk array. In specific embodiments, the machine-readable medium is more than one network.

The term “spectrum” is used herein to describe the range of visible wavelengths/frequencies covered in a defined camera range. For example, a visible spectrum is the captured image of an object as defined by wavelengths/frequencies in the visible light range of electromagnetic radiation. Alternatively, a thermal spectrum is the captured image of the electromagnetic radiation emitted from an object as defined by wavelengths/frequencies in the infrared range of electromagnetic radiation.

The term “synchronous” is used herein to describe the characteristic of associating initiating actions with other actions such that the initiating action is coordinated with subsequent actions in a defined sequence. In this way, the subsequent action is conditioned on the occurrence of a proceeding action that acts to initiate the subsequent action in a defined sequence. In certain embodiments, this synchronization is required based on the time constraints/limitations between the initiating action and the subsequent actions. For example, given that skin lesions cryogenically treated, e.g., with liquid nitrogen, are only visible for short window of time, i.e., in the range of seconds, the present invention requires synchronous capture of thermal and visible spectra, wherein the initiating action of the cryogenic treatment leads directly to the subsequent capture of thermal and visible spectra.

The language “user interface” is used herein to describe the graphical user interface (GUI), e.g., which allows a user to interface with the application programming interface (API), and enter data using interface components such as buttons, text fields, check boxes, etc.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . . and N, the text should be interpreted as requiring only one element from the group, not A plus N, or B plus N, etc.

What is claimed is:

1. A medical imaging and documentation system, comprising:
an image capture device configured to capture visible spectrum image data and thermal spectrum image data associated with a body surface of a patient;
a server comprising one or more processors configured to:
receive the visible spectrum image data and the thermal spectrum image dat;
detect a site of interest using at least one of the visible spectrum image data and the thermal spectrum image data;
map the site of interest to an anatomic coordinate space; and
store an anatomic localization record comprising stored anatomic coordinates for the site of interest.

2. The system of claim 1, wherein the image capture device comprises a thermal spectrum camera configured to capture the thermal spectrum image data and a visible spectrum camera configured to capture the visible spectrum image data.

3. The system of claim 1, wherein the server is further configured to transmit the anatomic coordinates for the site of interest to a medical record system to document the treatment locations in a patient record.

4. The system of claim 1, wherein the server is further configured to detect the site of interest by aggregating the visible spectrum image data with the thermal spectrum image data and mapping the aggregated image data to the anatomic coordinate space.

5. The system of claim 4, wherein the server aggregates the visible spectrum image data with the thermal spectrum image data to create a composite spectrum image, and wherein the server is configured to analyze the visible spectrum image data, the thermal spectrum image data, and/or the composite spectrum image to identify the site of interest.

6. The system of claim 5, wherein the server utilizes a machine learning model to analyze the visible spectrum image data, the thermal spectrum image data, and/or the composite spectrum image to identify the site of interest by:
identifying regions of the composite spectrum image or the thermal spectrum image data having a human body;
mapping each pixel of the regions to a specific surface part of the human body; and
detecting areas on the composite spectrum image or the thermal spectrum image data that are cooler than surrounding areas and assigning a location marker to each of the treatment areas, wherein the location marker maps to a portion of the human body.

7. The system of claim 4, further comprising:
an actuator configured to trigger the image capture device to capture the thermal spectrum image data and the visible spectrum image data.

8. The system of claim 1, wherein the image capture device is communicatively coupled with the server.

9. The system of claim 7, further comprising a housing configured to receive (i) the image capture device and hold the and (ii) a pressurized container configured to hold a cryogenic fluid.

10. The system of claim 9, wherein the actuator is configured to trigger the the image capture device to capture the thermal spectrum image data and the visible spectrum image data at a preset time after some of the cryogenic fluid is expelled from the pressurized container.

11. The system of claim 1, further comprising a medical notation API configured to interface the server with a database of the medical record system.

12. A computer-implemented method for detecting a site of interest on a body surface of a patient, comprising:
receiving first image data depicting the body surface of the patient during a first patient encounter;
determining a site representation corresponding to the site of interest in the first image data;
mapping the site representation to an anatomic coordinate space to determine anatomic coordinates for the site of interest;
storing the anatomic coordinates of the site of interest in a patient record;
receiving second image data depicting the body surface of the patient during a second patient encounter after the first patient encounter;
mapping the second image data to the anatomic coordinate space;
determining, based on the anatomic coordinates for the site of interest and the mapping of the second image data to the anatomic coordinate space, a current image location corresponding to the site of interest; and
displaying information of the site of interest.

13. The method of claim 12, wherein the first image data comprises visible spectrum image data and thermal spectrum image data captured during or after a cryotherapy procedure.

14. The method of claim 13, wherein the thermal spectrum image data and the visible spectrum image data were taken synchronously.

15. The method of claim 13, wherein determining the site representation comprises detecting a cryotherapy treatment site from a thermal signature in the thermal spectrum image data.

16. The method of claim 12, wherein the anatomic coordinate space comprises a body region identifier and surface coordinates corresponding to a surface parameterization of a three-dimensional body model.

17. The method of claim 12, wherein mapping the site representation to the anatomic coordinate space comprises applying a dense body mapping model that maps pixels of the first image data to surface coordinates of a body model.

18. A non-transitory computer-readable medium storing instructions that, when executed by one or more processors, cause the one or more processors to perform operations comprising:
receiving a source image depicting a source region on a body surface of a patient;
receiving a target image depicting the body surface of the patient after capture of the source image;
determining image correspondences between the source image and the target image;
transferring the source region to the target image based on the image correspondences to determine a transferred region;
storing localization data that associates the source region with an anatomic coordinate space; and
causing display of the transferred region on the target image to guide re-identification of the source region.

19. The non-transitory computer-readable medium of claim 18, wherein determining the image correspondences comprises applying a dense image feature matching model having a coarse matching stage and a refinement stage.

20. The non-transitory computer-readable medium of claim 18, wherein the operations further comprise causing display of the transferred region on the target image to guide re-identification of the source region using a display or a light source.
